(19) United States
(12) Patent Application Publication
(10) Pub. No.: US 2013/0252278 A1
(43) Pub. Date: Sep. 26, 2013

(54) PROCESS OF APPLICATION PROCEDURES THROUGH CELL BIOLOGY FOR ANIMAL USE

(71) Applicant: BIO-BIOTECNOLOGIA EM REPRODUCAO ANIMAL LTDA., (US)

(72) Inventor: Luis Mauro Valadao Queiroz, Lago Sul. Brasilia (BR)

(73) Assignee: BIO-BIOTECNOLOGIA EM REPRODUCAO ANIMAL LTDA., LAGO SUL. BRASILIA (BR)

(21) Appl. No.: 13/748,812

(22) Filed: Jan. 24, 2013

(30) Foreign Application Priority Data
Mar. 26, 2012 (BR) BR1020120074389

(51) Int. Cl. C12N 5/071

(52) U.S. Cl. CPC C12N 5/0653
USPC 435/40.5

(57) ABSTRACT

Process of application procedures through cell biology for animal use, the invention is applied to the field of cell biology, specifically the isolation, in vitro culture, cryopreservation, cell differentiation and application of mesenchymal stem cells derived from adipose tissue of animals for autologous cell therapy purposes. Strategically seeks to establish, from the adipose tissue collection, all events resulting in schematic form, in order to obtain a homogeneous extract of mesenchymal stem cells. In this invention, it is proposed a dynamic which allows the use of mesenchymal stem cells in veterinary cell therapy found in centers distant from the cell culture laboratory, since the transportation time of tissue and mesenchymal stem cells does not exceeds 78 hours after the their processing.

### CROSS-REFERENCE TO RELATED APPLICATIONS

This application claims priority under Brazil Application No. 1020 120074389 filed Mar. 26, 2012, herein incorporated by reference in its entirety.

### SHORT PRESENTATION

The present Invention Patent application relates to a process of application procedures through cell biology for animal use, which takes into account the fact that stem cells are, by definition, considered primitive cells capable of self-renewal and to differentiate into specialized cells from different tissues and is therefore considered totipotent or multipotent. These cells are found in the early stages of the embryo (embryo button), the fetus (mesoderm), newborn (umbilical cord) and adult (at different sites such as bone marrow, adipose tissue, dental pulp).

### FIELD OF APPLICATION

The invention is applied to the field of cell biology, specifically the isolation, in vitro culture, cryopreservation, cell differentiation and application of mesenchymal stem cells derived from adipose tissue of animals for autologous cell therapy.

The invention seeks to strategically establish, from the adipose tissue collection, all events resulting in schematic form, in order to obtain a homogeneous extract from mesenchymal stem cells.

In this invention, it is proposed a dynamic which allows the use of mesenchymal stem cells in veterinary cell therapy located in centers distant from the cell culture laboratory, since the transportation time of the tissue and mesenchymal stem cells does not exceeds 78 hours after the their processing.

### BACKGROUND OF THE INVENTION

Stem cells are, by definition, considered primitive stem cells capable of self-renewal and to differentiate into specialized cells from different tissues and is therefore considered totipotent or multipotent. These cells are found in the early stages of the embryo (embryo button), the fetus (mesoderm), newborn (umbilical cord) and adult (at different sites such as bone marrow, adipose tissue, dental pulp) (Strem B M et al, 2005; . . . Zuk P A et al, 2002; Oedayrajsingh Metal, 2007).

The conceptual difference between totipotency and multipotency cell is related to the ability of cells to vary in a wide variety of specialized cells, being totipotent cells those which show a higher capacity plastic to specialize. The totipotent cells are found in the embryo until the 6^{th} day (compact morula stage), between the 7^{th} and 14^{th} days of the embryo, in the cells that make up the embryo button.

The fetal cells found in the mesoderm, although having a high capacity for in vitro cell differentiation, have lines of formation and destination (thoraco-abdominal organs, bone, bone marrow, tendon, striated muscle, joint, adipose tissue) than those cells that form other fetal germ layers ectoderm (skin and nervous system) and endoderm (digestive tract).

Since the 1990s, several studies have characterized structurally and functionally the cells present in adults in different tissues (bone marrow, adipose, dental pulp) as mesenchymal stem cells (B M Strem et al, 2005; . . . Zuk PA et al, 2002; Oedayrajsingh M et al, 2007). This description is given by the presence of proteins (CD34 and STRO-1, for instance) of the plasma membrane only found in fetal mesenchymal cells. These cells have the capacity to differentiate in vitro, when chemically induced, and in vivo, into specialized cells, e.g., osteocytes, hepatocytes, cardiac and skeletal muscle fiber, tendon fibers, chondrocytes, adipocytes, among others. (Pittenger, M. F. et al. 1999; Zuk et al. 2001; Kotton, D. N. et al. 2001; Planat-Benard, V. et al. 2004; Long X et al. 2005; Freisinger E et al. 2008; Neupane, M. et al. 2008; Ryu, H. H. et al. 2009)

After the consolidation of these concepts, the establishment of techniques for isolation of mesenchymal stem cells from adults for the purpose of therapeutic applications has attracted the efforts of the scientific community.

Comparison of the plasticity and differentiation of mesenchymal stem cells found in different tissues of adult individuals, showed that there are functional similarities between the mesenchymal stem cells from bone marrow and adipose tissue. The identification of membrane proteins specific for cells of mesoderm, as well as the induction of cell differentiation to chondrocytes, osteocytes and adipocytes shows similar results when analysis is made for the mesenchymal stem cells derived from adipose tissue and bone marrow. (Zuk et al. In 2001; Puetzer, J L et al. In 2010, Placzek, M R 2009; Panetts, N J 2009)

In the laboratory routine, the establishment of a consistent isolation, culture and cryopreservation protocol on mesenchymal stem cells for the autologous cell therapy, takes into consideration other parameters, such as accessibility of the tissue source of stem cells, cell number available, cell concentration and rate of cellular homology (purity level).

The routine laboratory expertise for the isolation and characterization of mesenchymal stem cells shows a series of advantages to the use of adipose tissue as a source autologous cell, e.g., ease of access to adipose tissue, adipose tissue availability in an adult organism, concentration of mesenchymal stem cells into the interstitium of adipose tissue, cellular differentiation capacity in vitro (Zuk, P et al. 2001).

The method has disadvantages:
A. Method for transporting the adipose tissue from the operating room to the laboratory. There is no clear evidence in the literature for a maximum timeframe, without loss of the material viability;
B. The heterogeneity of the cell population obtained and the presence of debris at the end of the procedures for cell isolation;
C. Method for transporting mesenchymal stem cells from the laboratory to the operating room. There is no clear evidence in the literature for a maximum timeframe, without loss of the material viability.

### State of the Art

Stem cells are, by definition, considered primitive stem cells capable of self-renewal and to differentiate into specialized cells from different tissues and therefore is considered totipotent or multipotent. Until the 1990s, stem cells have been identified and isolated from embryonic and fetal tissues.

Pittenger et al (1999) characterized a homogeneous population of mesenchymal cells isolated from bone marrow from the iliac crest in humans. These mesenchymal cells were characterized by their ability to proliferate in vitro while retaining their morphology, by the presence of a set of plasma membrane protein markers specific for mesenchymal cells (SH2, SH3, CD34 and CD45) and by its ability to cell differentiation for multiple mesenchymal lineages (adipogenic, osteogenic and chondrogenic lineages) when stimulated by specific chemical inducers. (Pittenger, M. F. et al. 1999. Multilineage Potential of Adult Human Mesenchymal Stem Cells. Science, 284, p. 143-147)

In 2001, Zuk et al. reported the presence of mesenchymal stem cells in the human adipose tissue. Just as in the paper presented by Pittenger, these cells were isolated from the originating tissue and its ability to retain the morphology and function were evaluated after a long-term in vitro culture. The mesenchymal stem cells obtained from adipose tissue showed the same pattern of plasma membrane proteins specific to fetal mesenchymal cells. It also showed the ability of cellular differentiation to specialized chondrogenic, adipogenic and osteogenic cells when chemically induced during in vitro culture. (Zuk et al. 2001 Multilineage cells from human adipose tissue: Implications for cell-based Therapies. Tissue Eng, 7, 211-228).

In the following year, Zuk et al. (2002) confirmed the results previously reported for presenting mesenchymal stem cells derived from adipose tissue a wider range of cell differentiation chemically induced in vitro. In this paper, these cells were differentiated for myogenic and neurogenic lineages, besides, of course, the chondrogenic, adipogenic and osteogenic lineages. Moreover, histological, immunocytochemistry and indirect immunofluorescence analysis were related proving the structural and functional similarity of mesenchymal stem cells found in adipose tissue and those found in embryonic mesodermal layer.

Mizuno, H. and Hyakusoku, H. (2003) described, confirming the results obtained by Zuk et al. (2001) and Zuk et al. (2002), the ability of mesenchymal stem cells derived from adipose tissue to differentiate, in vitro, into specialized cells of chondrogenic, adipogenic, osteogenic and myogenic lineages. In this paper, the authors characterized by the analysis of intracellular b-galactosidase absence, the low standard of senescence of these cells, which is consistent with the low senescence characteristic of cells that make up the embryonic mesodermal tissue. (Mizuno, H. and Hyakusoku, H. 2003 Mesengenic and future clinical potential of human perspective lipoaspirate processed cells. Nippon J Med Sch, 70, p 300-306).

Since then, several studies have been developed and published in order to use these mesenchymal stem cells found in different sites of adult autologous cell therapy. This methodology seeks to regenerate damaged tissue without its partial or total loss of function.

By the early 2000s, it was believed that the mesenchymal stem cells had the ability to differentiate into tissue structures derived from the same embryonic germ laminate layer, the mesoderm. However, Kotton, D. N. et al. 2001 demonstrated the ability of mesenchymal stem cells from mouse bone marrow to differentiate into type I pneumocytes of the alveolar epithelial cells that make up the lung parenchyma. (Kotton, D N et al. 2001 Bone marrow-derived cells to progenitors of lung alveolar epithelium. Development, 128, p. 5181-5188).

Kang et al. 2004 compared the differentiation potential of mesenchymal stem cells obtained from adipose tissue and bone marrow from nonhuman primates to specialized cells of neurogenic lineage. The results suggest the ability of mesenchymal stem cells from both sources to differentiate through the use of chemical inducers in vitro culture (neurobasal medium added with B27, bFGF and EGF). The results suggest an increased potential for neurogenic mesenchymal stem cells derived from adipose tissue when compared to the neurogenic potential obtained by mesenchymal stem cells obtained from bone marrow. (Kang et al. 2004 Neurogenesis of rhesus adipose stromal cells. Journal of Cell Science 117, p. 4289-4299).

In agreement with these findings, Long X. et al. (2005) described the ability of these mesenchymal stem cells derived from adult bone marrow to differentiate into neuronal cells by chemical induction during in vitro culture. (Long X et al. 2005 Neural cell differentiation in vitro from adult human bone marrow mesenchymal stem cells. Stem Cells Dev, 14, p. 65-69).

Ryu, H. H. et al. (2009) demonstrated the increase in the neurological function in dogs with spinal cord injury after the application of mesenchymal stem cells from autologous adipose tissue. The authors suggest the autocrine and paracrine action of these cells in the lesion site, through the production of angiogenic, chemokines and anti-apoptotic cytokines, which would aid in the recruitment, proliferation and differentiation of neurogenic progenitor cells found near the injury site. (Ryu, H. H. 2009 Functional recovery and neural differentiation after transplantation of allogenic adipose-derived stem cells in a canine model of acute spinal cord injury. J. Vet. Sci. 10, p. 273-284).

In 2008, Freisinger E et al. reported the ability of mesenchymal stem cells isolated from human adipose tissue to differentiate into hematopoietic cells (macrophages) by monothioglycerol induction (MTG), interleukin 1-b and interleukin 3 and M-CSF.

Planat-Benard, V. et al. (2004), identified the presence in the adipose tissue of mesenchymal stem cells able to differentiate spontaneously to cardiomyocytes. In this work, the authors characterized these cells by morphological observations, confirmed by the presence of protein markers specific for cardiac fiber, immunocytochemical tests and ultrastructural analysis. Electrophysiological studies were also conducted, which revealed the presence of rhythmic and functional activities, when evaluating the response of these cells to adrenergic and cholinergic stimuli. (Planat-Benard, V. et al. 2004 Spontaneous Cardiomyocyte Differentiation From Adipose Tissue Stroma Cells. Circ Res. 94, p. 223-229).

Neupane, M. et al. (2008) suggested the use of a complex in vitro system to optimize the potential of cell differentiation and trophic effect of mesenchymal stem cells derived from adipose tissue of dogs. In this system, during the first 48 hours of in vitro culture, the D-MEM medium was used supplemented with N-acetyl-L-cysteine (NAC), L-ascorbic acid 2-phosphate, 10% fetal bovine serum and antibiotics. From the second day in culture, the medium used was the MCDB 153 supplemented with N-Acetyl-L-Cysteine (NAC), L-ascorbic acid 2-phosphate, 5% fetal bovine serum and antibiotics. (Neupane, M. et al. (2008) Isolation and characterization of canine adipose-derived mesenchymal stem cells. Tissue Engineering: Part A, 14, p.100′7-1015).

It is also known, from the prior art, the document U.S. Pat. No. 7,915,039, filed on Mar. 29, 2011, which provide a method for isolating mesenchymal stem cells from fetal tissue and adipose tissue in humans. According to the inventors, it is possible to obtain a population of mesenchymal stem cells with high homogeneity, since the method leads to tissue maceration and enzymatic treatment with collagenase solution in a MEM medium. The procedure seeks to remove the cells with the aid of the lysis solution and subsequent filtration of the processed tissue. For the filtration, two different filters are used sequentially with meshes of 100 μm and 10 μm. The authors describe in vitro cell culture in D-MEM medium plus 10% of FCS, for a period of 24 hours for identifying mesenchymal stem cells. This process differs greatly from that proposed in this document in two aspects. The first aspect is related to the use of D-MEM culture medium with the enzyme collagenase type I, where we describe the use of modified Dulbecco medium devoid of Ca and Mg, which facilitates the tissue breakdown and individualizing of the cells, without observing plasma membrane damage and subsequent cell loss. The second aspect refers to the order of the first filtration, where we suggest the filtration subsequent to incubation for 30 minutes of the homogenized tissue in a culture medium supplemented with the enzyme collagenase type I. Filtration now enables the early removal of debris and other unwanted tissue structures in the process of isolation of mesenchymal stem cells.

In the current state of the art, it is also known the document KR 20080007726, filed on Jan. 23, 2008, which discloses a method for culture of mesenchymal cells derived from umbilical cord blood in order to increase the rate of initial adherence of the mesenchymal stem cells derived of umbilical cord blood cells and embryonic stem cells, maintaining the multipotent characteristics for a long period of time. The culture in vitro method of mesenchymal stem cells derived from umbilical cord blood comprises the steps of: (a) prior coating of the cell culture bottle with gelatin, (b) inoculating the mesenchymal stem cells in the culture flask coated with gelatin, in an alpha-MEM culture medium and inactivated fetal bovine serum.

It is also known from the prior art, the document U.S. 2005118712, filed on Jun. 2, 2005, which presents a method for isolating mesenchymal stem cells of the amniotic fluid by a two phases protocol culture comprising culture of amniocytes (AFMSC) and of mesenchymal stem cells (MSC). For the culture of amniocytes, the primary cultures are created using the protocol of routine or standard in vitro culture in a cytogenetics laboratory. The non-adherent amniotic cells are collected in suspension in the amniotic fluid. For the culture of AFMSC cells, the amniotic fluid is centrifuged and the cells are inoculated on a plate obtained with alpha-MEM medium supplemented with fetal bovine serum. Analysis of the RT-PCR and immunohistochemistry showed that the mRNA for Oct-4 and its expression (Prot. Oct-4) are detectable in amniocytes (AFMSCs). When induced to differentiate in vitro, the AFMSCs can be differentiated into cells of various lineages, such as adipocytes, osteocytes and neuronal cells.

It is also known from the prior art the document U.S. 20040101959, filed on May 27, 2004, in which the inventors disclose a method for isolating mesenchymal stem cells, keratinocytes and fibroblasts derived from various tissues such as connective tissue, palate, skin, lamina propria, bone marrow and adipose tissue. The inventors recommend the use of extracellular matrix composed of biodegradable components of a series of cell activation, such as ascorbyl palmitate, linoleic acid, coenzyme Q-10, lipoic acid, calcium trifosfosadipose tissueo, calcium monophosphate, collagen, glycosaminoglycans, gelatin, polyglycolic acid, demineralized bone, hydroxyapatite, inorganic bone and coral, among others. For having characteristics of biodegradability, such extracellular matrix may be applied at the tissue injury site, or in adjacent regions. The inventors relates to mesenchymal stem cells, fibroblasts and keratinocytes with low energy laser light. The in vitro is done by free specific medium of immunogenic proteins present in the serum. In this document, the inventors recommend the use of this methodology in the treatment of periodontal defects and tooth, degenerative lesions of the skin and bone fracture repair in humans.

The patent PI0502668-7A describes a method of obtaining stem cells from mammalian tooth pulp. The authors describe the ability of these cells to differentiate in vitro and in vivo, thus being suitable for cell therapy processes, implemented for use in different areas of biotechnology, such as germplasm bank, gene therapy and tissue engineering. The process described by the inventors restricted to obtain stem cells from that of the dental pulp, according to these, with high purity in the population of cells cultured for a period of 1-3 weeks, keeping its characteristics of embryonic stem cells, pluripotent such as the capacity for self-renewal and continuous proliferation. Such period required to achieve the necessary population for cell therapy is too large since, for cell therapy to be successful, it must initiate the process in the acute phase of the lesion, i.e. the shortest possible period between the time of the lesion and the application of the cells. Thus, it is necessary to use a methodology that allows the isolation of stem cells with high purity, preserving the characteristics of pluripotency.

The methodology proposed in this paper allows up to 04 hours from the collection of adipose tissue, the isolation with a high degree of purity of mesenchymal stem cells, derived from this tissue, with characteristics of pluripotency, i.e. able to differentiate in vitro into specialized cells from the use of specific inducers. This feature allows immediate use in procedures for cell therapy, which ensures the short time necessary for treatment.

Taking into account the continental characteristics of Brazil, the inventors have a system of culture medium for the long transportation for the adipose tissue (48 hours) and to transport the mesenchymal stem cells (30 hours) to be applied on the focus of the lesion. The culture medium developed preserve desired characteristics of pluripotent mesenchymal stem cells. This process facilitates the logistics of sending and receiving adipose tissue stem cells by the veterinary practitioners, as shipping can be done by commercial companies specializing in express delivery. Thus, the action range of the cell culture laboratory extends up to the remote areas that have commercial air transport.

The Invention The development of the transport protocol of adipose tissue and mesenchymal stem cells for a period of up to 78 hours after the collection of adipose tissue and the application of mesenchymal stem cells isolated, which makes it possible to serve a continental radius around the Laboratory of Cell Culture, without loss of quality of material processed.

The technical result of the invention also shows the system for isolating mesenchymal stem cells derived from adipose tissue through a membrane filter with different pores, suitable for obtaining the cell extract to final processing laboratory. The unwanted presence of a heterogeneous population of cellular and tissue debris, reduce the quality of in vitro culture of mesenchymal stem cells and the result of application of stem cells on tissue injury focus.

With the present patent application, it is possible:
A. To transport to the Cell Culture Laboratory the sample of adipose tissue for up to 48 hours after its collection, without incurring any losses in the viability of the material.
B. During processing of the adipose tissue for the isolation of mesenchymal stem cells, using a filtration system serial, it has become possible to obtain an homogeneous extract of mesenchymal stem cells and without the presence of tissue debris.
C. To transport from the Cell Culture Laboratory to the surgical center (the site of application of the cells) for up to 30 hours, without incurring deleterious effects on the quality of the cells.

### DETAILED DESCRIPTION OF THE INVENTION

The process of application procedures through cell biology for animal use, application object of Invention Patent, consists of the following steps:

### A. Preparation of Media for Collection of Adipose Tissue

A.1. The preparation of Media for Collection of Adipose Tissue should be made on the day of sending these to the veterinarian who will collect them. The expiration date of this medium after its manufacture is 30 days.

A.2. For each collection of adipose tissue, a tube called Transport and other two tubes referred WASH #1 and WASH #2 should be fabricated.

According to the invention, the transport medium refers to Dulbecco's Modified medium—Phosphate Buffered Solution (DM-PBS) supplemented with fetal bovine serum at concentrations, which may range from 1% to 20%, pyruvic acid, with concentration, which may vary from 0.0022% to 0.0044%, glucose, which may vary with concentration from 0.05% to 0.15%, with cysteine concentration, which may range from 0.1 mM to 100 mM, epidermal growth factor (EGF) with a concentration which can vary from 0.1 ug % to 1.5 ug % penicillin and streptomycin antibiotics at concentrations 10.000 UI % to 10 mg %, with amphotericin concentration of phenol red 25 ug % and with concentration of 1 mg %.

The Wash #1 and Wash #2 media, themselves, refer to Dulbecco's Modified medium—Phosphate Buffered Solution (DM-PBS) supplemented with fetal bovine serum at concentrations which may range from 1% to 20%, penicillin, and antibacterial streptomycin, antifungal and phenol red at concentrations similar to those described for the transport means.

A.3. Preparation of Media for Collection of Adipose Tissue to be held in Laminar Flow, with the bench previously cleaned with 70% alcohol.

A.4. Place into the rack the amount of 50 ml conical tubes needed for the preparation of the media. Identify each conical tube with the names of resources that will be made, namely, “TRANSPORTATION”, “WASH #1” and “WASH #2.”

A.5. With the aid of a 20 ml syringe and 40×12 mm needle, add 35.0 ml of DM-PBS medium in each conical tube.

A.6. For the transport media, add bovine fetal serum, pyruvic acid, glucose, antioxidants, epidermal growth factor (EGF), antibacterial, antifungal and phenol red.

A.6. For the Wash #1 and Wash #2 media, add fetal bovine serum, antibacterial, antifungal and phenol red.

A.7. Seal the tubes with Parafilm.

### B. Kit Mounting for the Adipose Tissue Collection

B.1. The kit mounting for the Adipose Tissue collection should be made on the day of its shipment to the veterinarian who will collect it.

B.2. With the thermal insulated box, sanitize the interior with 70% alcohol.

B.3. Place in a rack the conical tubes called “TRANSPORTATION”, “WASH #1” and “WASH #2.”

B.4. Place, inside the thermal insulated box, the shelf with the conical tubes, two strips of Parafilm and ice packs. Fill in the blanks spots of the cooler with bubble wrap.

B.5. Close the cooler and seal with tape.

B.6. Fill in the draft order with the air data sender and the recipient.

C. Preparation of Media for Isolation of Stem Cells from Adipose Tissue C.1. Preparation of Media for Collection of Adipose Tissue should be made the day of receipt of adipose tissue in the laboratory.

C.2. For each sample of adipose tissue, it should be made:
C.2.1. MSC-AD medium: 50 ml
C.2.2. Digest medium: 25 ml
C.2.3. Erythrolysis medium: 12 ml
C.2.4. Cell Transport Medium: 15 ml

C.3. Preparation of Media for Collection of Adipose Tissue to be held in Laminar Flow, with the bench previously cleaned with 70% alcohol.

### D. Preparation of the MSC-AD Media

According to the present invention, the MSC-Ad medium refers to culture medium in vitro of mesenchymal stem cells comprising the Dulbecco's Modified medium, Eagle's Medium (D-MEM) supplemented with fetal bovine serum at concentrations which may vary from 1% to 25%, with cysteine concentration which may range from 0.1 mM to 100 mM, penicillin and streptomycin antibiotics at concentrations 10.000 UI % to 10 mg %, amphotericin with concentration of phenol red 25 ug % and with concentration of 1 mg %, and pyruvic acid, non-essential amino acids and essential growth factors such as EGF, and antioxidants may be further added.

D.1. Place in a rack a 50 ml conical tube, identifying it with the name “MSC-AD” and the date of manufacture.

D.2. Thaw an aliquot of Fetal Bovine Serum in the greenhouse stabilization.

D.3. With the aid of a pipette pump and 25 ml disposable serological pipette, add the appropriate volume of MSC-Ad Base Medium in 50 ml conical tube labeled “MSC-AD” to the manipulation.

D.4. Using the automatic pipettors P1000, add the Fetal Bovine Serum in the conical tube with the MSC-Ad Medium Base.

D.5. Add the other aforementioned component, such as pyruvic acid, EGF, antioxidants, amino acids.

D.6. For a period of one hour, maintain the “MSC-Ad” tube with the cover half-threaded in the stabilization CO2 incubator.

### E. Preparation of the Digest Medium

According to the present invention, the Digest medium refers to the auxiliary media used for dissociation of tissue. This medium consists of Dulbecco's Modified Phosphate Buffered Solution medium without calcium and magnesium (DM-PBS W/O Ca and Mg) plus the type-I enzyme collagenase, which may vary with concentration from 0.05% to 0.12%.

E.1. Remove from the freezer the 50 ml conical tube containing 25 ml of Digest medium, keeping it in the stabilized incubator for 2 hours. Make sure the lid is closed.

### F. Preparation of the Erythrolysis

According to the present invention, Erythrolysis medium refers to auxiliary media used for the lysis of erythrocytes in the process of isolation of mesenchymal stem cells. This medium is a solution of ammonia hydrochloride with a concentration that can range from 0.84% to 0.87%.

F.1. Remove from the refrigerator the 50 ml conical tube labeled “Erythrolysis.” Place it in an appropriate rack in the laminar flow bench.

F.2. Place another rack in a 15 ml conical tube, identifying it with the name “Erythrolysis” and the date of manufacture.

F.3. With the aid of a pipette pump and a 10 ml disposable serological pipette, add 12.0 ml of the Erythrolysis Medium in the 15 ml conical tube labeled “Erythrolysis.” Close the lid, sealing it with Parafilm.

F.4. Keep the Erythrolysis conical tube for a period of two hours in the stabilization incubator.

### G. Preparation of the Transport Medium for Stem Cells

According to the present invention, the transport media for Stem Cell refers to the media used to maintain the cells during the transport between the laboratory and the location of the animal to be treated. This medium is composed by Tissue Culture Medium 199 (TCM 199) supplemented with Hank's salts, Hepes buffer, fetal bovine serum, with a concentration that may range from 1% to 25%, with cysteine concentration that may range from 1 mM to 10 mM, with cysteine concentration that may range from 0.1 mM to 100 mM, epidermal growth factor (EGF) with a concentration that may range from 0.1 μg % to 1.5 μg %, penicillin and streptomycin antibiotics at a concentration of 10 mg % and 10.000 UI %, with amphotericin concentration of phenol red 25 μg % and with concentration of 1 mg % and may be further added pyruvic acid, non-essential amino acids and essential growth factors such as EGF, and antioxidants.

G.1. Remove from the refrigerator the vial containing the TCM199 medium. Place it in the laminar flow bench.

G.2. Place in a rack a 15 ml conical tube, identifying it with the name “TR-MSC” and the date of manufacture.

G.3. With the aid of a pipette pump and a 10 ml disposable serological pipette, add the required amount of TCM199 medium in the 15 ml conical tube labeled “TR-MSC.”

G.4. Add the other aforementioned component, such as pyruvic acid, EGF, antioxidants, amino acids.

G.5. Close the lid, sealing it with Parafilm.

G.6. Keep the conical tube RT-MSC for a period of one hour in the stabilized incubator.

### H. Receiving, Cleaning and Soaking of the Adipose Tissue

H.1. All steps in between receiving the transport tube containing the adipose tissue to the filling of stem cells in the syringe and/or the beginning of in vitro culture should be performed in Laminar Flow, with the bench previously cleaned with 70% alcohol.

I. Reception of the Transport Tube Containing the Adipose Tissue

I.1. In the anteroom of the Stem Cells Laboratory, open the thermal box containing the tube of Transport with the adipose tissue. Remove the tube and check the conditions of the submitted material (size of adipose tissue, temperature, cleanliness of the bottle, presence of debris and blood). Do a cleanup of the outside wall of the Transportation tube with 70% alcohol and a paper towel.

1.2. In laminar flow, position in an appropriate rack the transport tube. In the same rack, place another three 50 ml conical tubes.

1.3. With the aid of a 20 ml syringe and 40×12 needle disposable, remove 60 ml of DM-PBS solution and transfer 20 ml of this solution into each of the conical tubes.

1.4. With a sterile hemostat tweezers, remove the adipose tissue from the transportation tube and transfer to the tube containing the DM-PBS solution. Close the tube and mix the DM-PBS solution carefully with adipose tissue in an attempt to remove as much of blood and debris. Repeat this process in the other two conical tubes containing the DM-PBS solution.

1.5. At the end of this process, transfer the tissue to a sterile 100×20 petri plate.

### J. Digestion of the Adipose Tissue

J.1. With the aid of a sterile scalpel and hemostat tweezers, cut the adipose tissue into small pieces (up to 2 mm). Remove fascia and other unwanted tissues, if any.

J.2. Carefully transfer macerated adipose tissue to the tube containing 25 ml of stabilized Digest medium.

J.3. Return the Digest tube, now with the adipose tissue, to the stabilization incubator, maintaining it for a period of 30 minutes.

J.4. Thereafter, remove the Digest tube from the stabilization incubator and place in a suitable rack. Add 20 ml of MSC-Ad medium. Homogenize the MSC-Ad medium with the Digest medium and the adipose tissue.

J.5. In the same shelf, place a sterile 50 ml conical tube. Open the lid and place a collector filter with pores of 100 ul on it.

J.6. Carefully transfers the contents of the tube containing the adipose tissue to the collector filter. Work through the passage of the solution by the collector filter screen. At the end of the passage, check the volume of the 50 ml conical tube under the collector filter.

J.7. With the aid of a pipette pump and 10 ml disposable serological pipette, transfer to the collector filter the volume of the MSC-Ad medium required to complete the 50 ml conical tube that is positioned under it.

J.8. Follow the whole passage of the MSC-Ad medium through the filter into the conical tube.

J.9. Discard the collector filter and cover the 50 ml conical tube.

J.10. With the aid of the pipette pump and a 10 ml disposable serological pipette, distill in equal volumes the contents of the 50 ml conical tube in four 15-ml conical tubes.

J.11. Properly position the 4 conical tubes in a 15 mL centrifuge. Start the centrifuge at a speed of 1250 RPM for a period of 9 minutes.

### K. Erythrolysis

K.1. Thereafter, remove the 4 conical tubes from the centrifuge and place them in an appropriate rack in the laminar flow.

K.2. Place a fifth 15 ml conical tube in the rack with the other.

K.3. With the aid of a pipette pump and 10 ml disposable serological pipette, carefully remove all the supernatant of the 4 conical tubes. Dispose it.

K.4. Re-suspend the pellet in each tube with 3 ml of the stabilized Erythrolysis medium.

K.5. Again, with the aid of the pipette pump and a 10 ml disposable serological pipette, transfer the contents of each of four conical tubes to the fifth tube, already positioned on the rack.

K.6. Place this tube in the centrifuge cone. Start the centrifuge at a speed of 1250 RPM for a period of 5 minutes.

### L. Washing of Stem Cells and Removal of Debris

L.1. Thereafter, remove the conical centrifuge tube and place it in a suitable rack in laminar flow.

L.2. With the aid of the pipette pump and a 5 ml disposable serological pipette, carefully remove the supernatant from the conical tube. Dispose it.

L.3. With the aid of a pipette pump and 5 ml disposable serological pipette, re-suspend the pellet of the tube with 4 ml of stabilized TR-MSC medium.

L.4. Place this conical tube in the centrifuge. Start the centrifuge at a speed of 1250 RPM for a period of 3 minutes.

L.5. In the same shelf, place a sterile 15 ml conical tube. Open the lid and place it on the system of filters with pores of 80 ul and 10 ul.

L.6. Carefully transfers the contents of the tube containing the adipose tissue for the filter system. Work through the passage of the solution through the screens of the filter system. At the end of the passage, rinse the filter system with more 4 ml of TR-MSC medium.

L.7. Repeat procedures K.1. to K.6. two more times.

### M. Filling Stem Cells to 1.0 mL Syringe

M.1. Thereafter, remove the conical tube from the centrifuge and place it in a suitable rack in laminar flow.

M.2. With the aid of the pipette pump and a 5 ml disposable serological pipette, carefully remove the supernatant from the conical tube. Dispose it.

M.3. Position 2 of 1.8 ml cryovials in a suitable rack. With the aid of an automatic pipettor P1000, transfer 1.5 ml of TR-MSC medium for each cryovial.

M.4. With the aid of an automatic pipettor P1000, re-suspend the pellet with 1.0 ml of TR-MSC medium.

M.5. With the same pipettor, homogenize the medium with the cells. Remove 0.3 ml of volume transfer and to each of 1.8 ml cryotubes. Save the remaining volume in the tube. Keep it on a proper shelf.

M.6. Homogenize the content (TR-MSC medium and Stem Cells) of cryotubes.

M.7. Attach carefully six 30×8 needles in six 1.0 ml syringes.

M.8. Performa filling of 0.5 ml cryotubes contents of the 1.0 ml syringes.

M.9. After the filling of these syringes, put them inside a plastic bag 20×8 cm.

M.10. Seal the plastic bag containing syringes and carefully place it in a styrofoam box of 2 lts. Fill the empty spaces of the Styrofoam box with bubble wrap.

### N. Initiation of in Vitro Culture of Stem Cells

N.1. Place upright two 25 cm² bottles of tissue culture in laminar flow.

N.2. With a permanent marker, identify the external and bottom side wall of each of the cylinders, the animal's name (or veterinary), the manipulation data, the identification ROCO and animal species.

N.3. With the aid of the pipette pump and a 10 ml disposable serological pipette, transfer 7.5 ml of the MSC-Ad medium into each cell culture bottle.

N.4. With the aid of an automatic pipettor P1000, gently mix the contents of the 15 ml conical tube saved in Item F.4.

N.5. Transfer in equal parts the contents of two bottles of cell culture. Close the lid of each bottle.

N.6. Analyze the cell characteristics in inverted microscope with Hoffmann contrast.

N.7. Transfer the cell culture bottles for cell culture incubator.

N.8. Keep them with lids half-open.

### O. Feeding of the In Vitro Culture

O.1. The feeding of the cell culture should be performed every two days from the beginning of cell culture.

O.2. The stem cell cultures can reach the stage of cell confluence than 90%.

O.3. All actions related to the feeding stage of in vitro culture should be performed in Laminar Flow, with the bench previously cleaned with 70% alcohol.

O.4. Remove the cell culture bottle of CO2 incubator, previously closed with the lid, position in lying down in a laminar flow bench.

O.5. Place in a suitable rack the MSC-Ad medium tube. 0.6. With the aid of a pipette pump and disposable 10 ml serological pipette, carefully remove all MSC-Ad medium contained in the cell culture bottle. Neglect the content. Be careful not to touch the bottom of the cell culture plate with the serological pipette tip.

O.7. With the aid of the pipette pump and a new 10 ml disposable serological pipette, add 8.0 ml of MSC-Ad medium for the cell culture bottle.

O.8. Close the lid and place the cell culture bottle so that the cell layer is submerged in MSC-Ad medium.

O.9. Return the cell culture bottle for the culture incubator.

O.10. Keep it with the lid half open.

### P. Preparation of Material for Freezing

P.1. Stabilization of cell Freezer.
P.1.1. This is a polystyrene box of 7 liters, with a shelf (as a frame) of 20×12×4 cm.
P.1.2. Transfer the liquid nitrogen of the nitrogen cylinder to freeze the cells until the specified time (about 60% of internal volume).
P.1.3. Close the freezer with the cover. After 20 minutes, check the level of liquid nitrogen. If it is below the identified mark, complete with nitrogen up to the mark.

P.2. Labelling of the vials.
P.2.1. Turn the labeler and check the battery charge level and the amount of label on the tape cartridge.
P.2.2. Enter the animal data and the procedure in the following order: Identification of the animal (name or RGD), species, date of freezing.
P.2.3. Print the number of labels corresponding to the number of vials to be frozen.
P.2.4. On each label, cut the side edges and bottom.
P.2.5. In laminar flow bench, remove from the package the required amount of vials. Label each vane carefully to avoid handling the end that does not contain the bushing.
P.2.6. For each label, remove its protective role, and near the end of the vial with the bushing, secure, first, its upper edge. Then screw the rest of the label.

### Q. Rebound of Stem Cells

Q.1. The subculture (trypsinization) of stem cells in vitro culture should strategically place until you get a confluence in cell culture bottle of 90%.

Q.2. All actions related to the rebound step in vitro culture should be performed in Laminar Flow, with the bench previously cleaned with 70% alcohol.

Q.3. Stabilize means to be used solution (Trypsin/EDTA solution, MSC-Ad medium, CRYO-MSC-Ad medium and Fetal Bovine Serum), at least one hour before the start of the activities of rebound and freezing, packing them in the stabilization incubator.

Q.4. According to the present invention, the CRYO-MSC-Ad medium refers to Dulbecco's Modified Eagle's Medium (D-MEM) supplemented with fetal bovine serum at concentrations which may range from 10% to 30% dimethylsulfoxide (DMSO), with a concentration which can range from 10 to 20%, penicillin and streptomycin antibiotics at concentrations 10.000 UI % to 10 mg %, with amphotericin concentration of phenol red 25 ug % and with concentration of 1 mg % and may be further increased pyruvic acid, essential amino-acids and non-essential growth factors such as EGF, and antioxidants.

Q.4. Remove the cell culture bottle of the CO2 incubator, previously closed with the lid, positioning it lying down in a laminar flow bench.

Q.5. Place in a suitable rack tubes with the trypsin/EDTA solution and Fetal Bovine Serum.

Q.6. With the aid of a pipette pump and disposable 10 ml serological pipette, carefully remove all MSC-Ad medium contained in the cell culture bottle. Neglect the content. Be careful not to touch the bottom of the cell culture plate with the serological pipette tip.

Q.7. With the aid of the pipette pump serological pipette and a new disposable 10 ml, add 5.0 ml of the Trpsina/EDTA solution into the cell culture bottle.

Q.8. Close the lid and place the cell culture bottle so that the cell layer is submerged in the Trpsina/EDTA solution.

Q.9. Return the cell culture bottle to the culture incubator, holding it there for a period of 2 minutes. Q.10. Assess under ambient light the level of detachment of cells from the cell culture plate.

Q.11. Confirm this assessment in inverted microscope with Hoffmann contrast. Q.12. If you notice that almost all the stem cells came off the cell culture plate, return it to the laminar flow bench. If there is still a high number of cells adhered to the back plate to the stove, keeping it for another 30 seconds. Repeat the P.9, P.10 and P.11.

Q.13. Place the cell culture bottle upright, open the lid and, using a pipette pump and 5 ml disposable serological pipet, add 5.0 ml of Fetal Bovine Serum.

Q.14. Homogenize the content of the cell culture bottle.

Q.15. With the aid of the pipette pump and 5.0 ml disposable serological pipette, transfer the contents of the cell culture bottle to a 15 ml conical tube.

Q.16. Place the tube properly in 15 ml conical centrifuge. Start the centrifuge at a speed of 1250 RPM for a period of 2 minutes.

Q.17. Thereafter, remove the conical centrifuge tube and place in a suitable rack in laminar flow.

Q.18. With the aid of the pipette pump and disposable serological pipette 5.0 ml, carefully remove all the supernatant of the conical tube. Dispose it.

Q.19. Re-suspend the pellet of the conical tube with 5 ml of CRYO- MSC-Ad medium.

Q.20. Homogenize.

Q.21. Attach properly the end of the labeled vial containing the bushing adapter of the filling syringe.

Q.22. With the other end of the vial immersed in the CRYO-MSC-Ad medium+stem cells, and with the aid of the filling syringe, aspirate the contents filling the vial according to the drawing below. Note that the last column using CRYO-MSC-Ad+stem cells should soak the bushing.

Q.23. Disengage the vial of the filling syringe. Seal the free end of the vial with the proper seal.

Q.24. Repeat the procedures 6.1.20 to 6.1.22 for each of the labeled vials.

Q.25. Transfer all the vials potted to the shelf of the freezer cell.

Q.26. Open the lid of the freezer and quickly position the shelf with the vials on the liquid nitrogen freezer.

Q.27. Recap the freezer. Keep it closed for 20 minutes.

Q.28. Thereafter, open the freezer and quickly turn the shelf so that the vials fall directly into the liquid nitrogen.

Q.29. Immersed in liquid nitrogen and with the aid of tweezers, pack the straws containing the frozen stem cells identified in the appropriate racks.

Q.30. Transfer identified racks to the nitrogen cylinder. Write down the information regarding the location of the cylinder rack (cylinder identification and mug number).

Q.31. Enter this information on the location of these vials in the appropriate system.

### R. Heating the Water for Bath

R.1. With the aid of water heater and a container (500 ml), warm 500 ml of filtered water at a temperature of 37° C.

### S. Preparation of Cell Culture Bottles

S.1. Place two 25 cm² tissue culture bottles upright in laminar flow.

S.2. With a permanent marker, identify the external bottom and side wall of each of the cylinders, the animal's name (or veterinary), the manipulation date, the identification R1C1 and animal species.

S.3. With the aid of the pipette pump and a 10 ml disposable serological pipette, transfer 7.5 ml of the MSC-Ad medium into each cell culture bottles.

### T. Thawing of Stem Cells

T.1. All actions related to the subsequent steps of heating water for thawing of stem cells should be performed in Laminar Flow, with the bench previously cleaned with 70% alcohol.

T.2. Locate the rack containing the cells to be thawed. Check the data described in the rachis and the vial.

T.3. With tweezers, carefully remove a vial to be thawed from the racks.

T.4. Keep the vial in the air for 5 seconds. After that time, dip the vial in water heated to 37° C. Keep immersed in water for 10 seconds.

T.5. Remove the vial from the container thawed and dry thoroughly with paper towels.

T.6. With the aid of scissors cut off the end of the vial that contains the seal. Attach carefully this end to the filling syringe. Cut the other end of the vial (bushing).

T.7. Transfer the contents of the vial to a 15 ml conical tube containing 5.0 ml of MSC-Ad medium.

T.8. Place the tube properly in a 15 ml conical centrifuge. Start the centrifuge at a speed of 1250 RPM for a period of 2 minutes.

T.9. Thereafter, remove the conical centrifuge tube and place in a suitable rack in laminar flow.

T.10. With the aid of the pipette pump and 5.0 ml disposable serological pipette, carefully remove all the supernatant of the conical tube. Dispose it.

T.11. With the aid of an automatic pipettor P1000 carefully homogenize the contents of the conical tube. Transfer in equal parts the contents of two pre-prepared cell culture bottles. Close the lid of each bottle.

T.12. Analyze the cell characteristics in inverted microscope with Hoffmann contrast.

T.13. Transfer the cell culture bottles into the cell culture incubator.

T.14. Keep them with the lids half-open.

### U. Inducing Cell Differentiation—Osteogenic Lineage

U.1. The preparation of the means of stem cell differentiation (DIF medium) in osteoblast should be made where identified a cell confluence of 75 to 90% of in vitro culture bottle.

U.2. For every bottle of in vitro culture, it should be made up 10.0 ml of DIF Medium.

U.3. According to the present invention, DIF medium refers to medium of inducing the differentiation of mesenchymal stem cells for in vitro culture osteogenic lineage, which is composed by Dulbecco's Modified Eagle's Medium (D-MEM) supplemented with fetal bovine serum at concentrations which may range from 1% to 25%, with dexamethasone concentration which can range from 0.05 μM to 0.15 μM, beta-glycerophosphate may vary with concentration of 1 mM to 15 mM, ascorbic acid with a concentration which can vary 10 A of the 100 um, with cysteine concentration which may range from 0.1 mM to 100 mM, penicillin and streptomycin antibiotics at concentrations 10.000 UI % to 10 mg %, with amphotericin concentration of phenol red 2 5ug % and with concentration of 1 mg % and may be even added with pyruvic acid, non-essential amino acids and essential growth factors such as EGF, and antioxidants.

U.4. The preparation of DIF medium should be performed in Laminar Flow, with the bench previously cleaned with 70% alcohol. U.5. First, prepare the MSC-Ad medium, according to the instructions contained in the Operating Procedure P-003-ULAB. This medium will be the base medium for making the cell differentiation medium (DIF medium).

U.6. Thaw an aliquot of DIF1, DIF2 and DIF3.

U.7. With the aid of a pipette pump and a 10 ml disposable serological pipette, add 10.0 ml of MSC-Ad Base Medium in 15 ml conical tube labeled “DIF”.

U.8. Using automatic pipettors P20 and P200, add 10.0 μl of DIF1, 50.0 μl of DIF2 and 10.0 μl of DIF3 into the conical tube “DIF”.

U.9. For a period of one hour, keep the tube “DIF” with the lid half-screwed in the stabilization CO₂ incubator.

U.10. All actions related to stage of Cell Differentiation must be made in Laminar Flow, with the bench previously cleaned with 70% alcohol.

U.11. Remove the cell culture bottle of the CO₂ incubator, previously closed with the lid, positioned lying down in a laminar flow bench.

U.12. Place the tube with the DIF medium in a suitable rack.

U.13. With the aid of a pipette pump and 10 ml disposable serological pipette, carefully remove all MSC-Ad medium contained in the cell culture bottle. Neglect the content. Be careful not to touch the bottom of the cell culture plate with the serological pipette tip.

U.14. With the aid of the pipette pump and a new 10 ml disposable serological pipette, add 8.0 ml of DIF medium into the cell culture bottle.

U.15. Close the lid and place the cell culture bottle so that the cell layer is submerged in the MSC-Ad medium.

U.16. Return the cell culture bottle to the culture incubator.

U.18. Keep it with the lid half open.

U.19. Repeat this procedure every 48 hours for a period of 10 days.

### V. Specific Staining for Osteogenic Lineage Cells

V.1. All actions related to stage of cell differentiation should be carried out on a bench with sink.

V.2. Remove the cell culture bottle of the CO2 incubator, with the lid closed before. Check the inverted microscope and the confluence point of the cell culture.

V.3. Place the cell culture bottle on the counter near the sink.

V.4. In an appropriate rack, place the 15 ml conical tubes with 8.0 ml of the following solutions:
1. DM-PBS (2 tubes);
2. Formalin Solution 10%, it means 10% paraformaldehyde;
3. Deionized Water;
4. Osteoblast Coloring solution.

V.5. According to the present invention, Osteoblast Coloring solution refers to coloring specific solution for differentiated cells for osteogenic lineages for presenting a high concentration of calcium ion in the cytoplasm, and composed by alizarin 0.4%.

V.6. With the aid of a pipette pump and a 10 ml disposable serological pipette, carefully remove all medium present in the cell culture bottle. Neglect the content. Be careful not to touch the bottom of the cell culture plate with the serological pipette tip.

V.7. With the aid of the pipette pump and a new 10 ml disposable serological pipette, add 8.0 ml of DM-PBS medium in cell culture bottle.

V.8. Close the lid and place the cell culture bottle such that the cell layer is submerged in DM-PBS medium.

V.9. With the aid of a pipette pump and 10 ml disposable serological pipette, carefully remove all DM-PBS medium contained in the cell culture bottle. Neglect the content.

V.10. With the aid of the pipette pump and a new 10 ml disposable serological pipette, add 8.0 ml of 10% formalin solution in the cell culture bottle.

V.11. Close the lid and place the cell culture bottle such that the cell layer is submerged in the 10% formalin solution for a period of 30 minutes.

V.12. With the aid of a pipette pump and a 10 ml disposable serological pipette, carefully remove the entire 10% formalin solution contained in the cell culture bottle. Neglect the content.

V.13. With the aid of a pipette pump and a 10 ml disposable serological pipette, add 8.0 ml of the staining solution into cell culture bottle.

V.14. Close the lid and place the cell culture bottle such that the cell layer is submerged in the staining solution for a period of 10 minutes.

V.15. With the aid of a pipette pump and a 10 ml disposable serological pipette, carefully remove any staining solution contained in the cell culture bottle. Neglect the content.

V.16. With the aid of a pipette pump and a 10 ml disposable serological pipette, add 8.0 ml of Deionized water into the cell culture bottle.

V.17. With the aid of a pipette pump and 10 ml disposable serological pipette, carefully remove all Deionized water contained in the cell culture bottle. Neglect the content.

V.18. With the aid of a pipette pump and a 10 ml disposable serological pipette, add 8.0 ml of DM-PBS into the cell culture bottle.

V.19. Close the lid and place the cell culture bottle such that the cell layer is submerged in the cell DM-PBS.

V.20. Note the inverted microscope with a phase contrast for cells stained in red.

What is claimed:

1. Process of application procedures through cell biology for animal use, which applies to totipotent or multipotent cells, which are found in the early stages of the embryo (embryo button), the fetus (mesoderm), newborn (cord cord) and in adult individuals (at different sites such as bone marrow, adipose tissue, dental pulp, wherein the use of mesenchymal stem cells in cell therapy in veterinary laboratories, cell culture, establishing, from the collection of adipose tissue all events resulting in schematic form, in order to obtain a homogeneous extract from mesenchymal stem cells; applied to the isolation, in vitro culturing, cryopreservation, use of cell differentiation and mesenchymal stem cells from adipose tissue of animals for autologous cell therapy, and consists of the following steps: Preparation of Medium for Collection of Adipose Tissue, Mounting of the kit for Adipose Tissue Collection, Preparation of medium for isolation of stem cells from the Adipose Tissue; Preparation of the MSC-AD;
Preparation of the Digest medium; Preparation of the Erythrolysis medium; Reception, washing and maceration of adipose tissue; Reception of the transport tube containing the adipose tissue; Adipose tissue digestion; Erythrolysis; Drying of stem cells and removal of debris; Filling stem cells to a syringe of 1,0 ml; Initiation of in vitro culture of stem cells; Feeding the Growing In Vitro; Preparation Material for freezing; peal of stem cell; heating the water bath, preparation of cell culture bottles; Thawing of stem cells; Induction of cellular differentiation—Osteogenic lineage; Specific staining for osteogenic lineage cells.

2. Process of application procedures through cell biology for animal use, according to claim 1, wherein, during the processing of adipose tissue for the isolation of mesenchymal stem cells, it is obtained a homogeneous extract of mesenchymal stem cells without the presence of tissue debris.

3. Process of application procedures through cell biology for animal use, according to claim 1, wherein the preparation of the Medium for the Collection of Adipose Tissue is made on the day of sending these to the veterinarian who shall make the collection; for each collection of adipose tissue, are made through a tube called Transportation and two tubes called Wash #1 and Wash #2; the preparation of medium for the Collection of Adipose Tissue is made in Laminar Flow, with the first bench cleaned with 70% alcohol; place a rack in the amount of 50 ml conical tubes required for the preparation of medium and identify each conical tube with the names of the means to be made, namely, “TRANSPORTATION”, “WASH #1” and “WASH #2.”

4. Process of application procedures through cell biology for animal use, according to claims 1 and 3, wherein each set of three conical tubes, thawing two aliquots of Fetal Bovine Serum in the stabilization incubator and adding 35.0 ml of PBS in each conical tube with the PBS solution.

5. Process of application procedures through cell biology for animal use, according to claim 1 or 4, wherein it is added 500 μl of bovine fetal serum and 25 μl of Phenol Red solution, respectively, in tubes called “Wash #1” and “Wash #2”.

6. Process of application procedures through cell biology for animal use, according to claim 1 or 5, wherein it is added 1000 μl of bovine fetal serum and 50 μl of Phenol Red solution, respectively, in the tube called “Transportation”.

7. Process of application procedures through cell biology for animal use, according to claim 1 or 6, wherein the tubes are sealed with Parafilm.

8. Process of application procedures through cell biology for animal use, according to claim 1, wherein the mounting kit for the Collection of Adipose Tissue is made on the day of submitting this to the vet to carry out the collection in an thermal insulated box cleaned with alcohol 70%, with a rack positioned in the conical tubes called “TRANSPORTATION”, “WASH #1” and “WASH #2”, placing the shelf inside the thermal insulated box with the conical tubes, two strips of Parafilm and ice packs, whereby the empty spaces of the insulated box with protective material, the thermal box is closed and sealed, followed by clearance procedures.

9. Process of application procedures through cell biology for animal use, according to claim 1, wherein the preparation of medium for the Collection of Adipose Tissue is made on receipt of adipose tissue in the laboratory, for each collection of adipose tissue, should be made: MSC-AD Medium: 50 ml; Digest Medium: 25 ml; Erythrolysis Medium: 12 ml; Methods for preparation of Adipose Tissue Collection held in Laminar Flow, with the bench previously cleaned with 70% alcohol.

10. Process of application procedures through cell biology for animal use, according to claim 1, wherein the Preparation of the MSC-AD requires positioning the rack in a 50 ml conical tube, identifying it with the name “MSC-AD” and the date of manufacture and thawing of an aliquot with 5.0 mL of Fetal Bovine Serum in the greenhouse stabilization.

11. Process of application procedures through cell biology for animal use, according to claims 1 and 10, wherein it is added 45.0 ml of MSC-Ad Base Medium in 50 ml conical tube labeled “MSC-AD”.

12. Process of application procedures through cell biology for animal use, according to claim 1 or 11, wherein it is added 5.0 ml of Fetal Bovine Serum in the conical tube with the MSC-Ad Medium Base.

13. Process of application procedures through cell biology for animal use, according to claim 1, 10 or 12, wherein it is added 1000 μl of bovine fetal serum and 50 μl of Phenol Red solution, respectively, on the tube called “Transportation.”

14. Process of application procedures through cell biology for animal use, according to claim 1, 10 or 11, wherein, by during a period of one hour, keeping the tube “MSC-AD” with the lid half-threaded in the stabilization CO2 incubator.

15. Process of application procedures through cell biology for animal use, according to claim 1, wherein the Preparation of the Digest medium requires the withdrawal of the 50 ml conical tube from the freezer containing 25 ml of Digest medium, keeping in the stabilization incubator for 2 hours.

16. Process of application procedures through cell biology for animal use, according to claim 1, wherein the preparation of the Erythrolysis medium requires the removal of the 50 ml conical tube from the refrigerator labeled “Erythrolysis”, positioning it in an appropriate racks at the laminar flow bench.

17. Process of application procedures through cell biology for animal use, according to claims 1 and 16, wherein there are a positioning in another rack a 15 ml conical tube, identifying it with the name “Erythrolysis” and the date of the making it.

18. Process of application procedures through cell biology for animal use, according to claim 1 or 17, characterized by adding 12.0 ml of Erythrolysis medium in a 15 ml conical tube labeled “Erythrolysis”, the lid is closed and sealed with Parafilm, the Erythrolysis conical tube is tapered maintained for a period of two hours in the stabilization incubator.

19. Process of application procedures through cell biology for animal use, according to claim 1, wherein all the steps in between receive the Transport tube containing the adipose tissue to the filling of the stem cells in syringe and/or the beginning of in vitro culture being performed in Laminar Flow, with the bench previously cleaned with 70% alcohol.

20. Process of application procedures through cell biology for animal use, according to claim 1, wherein the reception of the transport tube contains the adipose tissue to be made in the anteroom of the Laboratory of Stem Cells, opening the Thermal Box containing the Transport tube in adipose tissue by removing and making sure the conditions of the submitted material (size of adipose tissue, temperature, cleanliness of the bottle, the presence of debris and blood), then proceeding the clean up on the outside wall of Transportation tube with 70% alcohol and a paper towel.

21. Process of application procedures through cell biology for animal use, according to claims 1 and 20, wherein, in the laminar flow, it is positioned a rack in the appropriate transport tube, and, in the same rack, positioning other three 50 ml conical tubes.

22. Process of application procedures through cell biology for animal use, according to claim 1, 20 or 21, wherein it is removed 60 ml of PBS solution and transfer 20 ml of this solution into each of the conical tubes.

23. Process of application procedures through cell biology for animal use, according to claim 1 or 22, wherein there are a removal of the adipose tissue of the transport tube and transfer to the tube containing the PBS solution; one should seal the tube and mix thoroughly the PBS solution with the adipose tissue removing as much of blood and debris; the operation is repeated for the other two conical tubes containing the PBS solution.

24. Process of application procedures through cell biology for animal use, according to claim 1 or 23, wherein there is a transferring of the tissue to a sterile petri dish.

25. Process of application procedures through cell biology for animal use, according to claim 1, wherein the digestion of adipose tissue is made by cutting the adipose tissue into small pieces (up to 2 mm), removing fascias and other tissues that are not allowed, if any.

26. Process of application procedures through cell biology for animal use, according to claim 1 or 25, wherein there is a transferring adipose tissue macerated in the tube containing 25 ml of stabilized Digest medium.

27. Process of application procedures through cell biology for animal use, according to claim 1 or 26, wherein there is a returning of the tube to digest adipose tissue, to the stabilized incubator, keeping it for a period 30 minutes.

28. Process of application procedures through cell biology for animal use, according to claim 1 or 27, wherein, in the end of the period, there is the removing of the Disgest tube from the stabilization incubator and positioning it into a rack.

29. Process of application procedures through cell biology for animal use, according to claim 1 or 28, wherein it is added 20 ml of MSC-Ad medium and mix the MSC-Ad medium with the Digest medium and the adipose tissue.

30. Process of application procedures through cell biology for animal use, according to claim 1 or 29, wherein, on the same shelf, it is placed a sterile 50 ml conical tube, opening the lid on it and placing a nylon collector filter (80 ul size pore), already without the sleeve and the lid; transferring the contents of the tube containing the adipose tissue to a collector filter;
at the end, checking the volume of the 50 ml conical tube under the collector filter;
transferring for the collector filter the volume of MSC-Ad medium required to complete the 50 ml conical tube which is positioned under it.

31. Process of application procedures through cell biology for animal use, according to claim 1 or 30, wherein there is a collector filter disposal and capping of the 50 ml conical tube.

32. Process of application procedures through cell biology for animal use, according to claim 1 or 30, wherein there is a fractionating in equal volumes of the contents of the 50 ml conical tubes in four 15 ml conical tubes, which are placed in a centrifuge.

33. Process of application procedures through cell biology for animal use, according to claim 1 or 32, wherein the erythrolysis is held at the end of this period, removing the four conical tubes from the centrifuge and positioning them in the appropriate rack in laminar flow.

34. Process of application procedures through cell biology for animal use, according to claim 1 or 33, wherein a fifth 15 ml conical tube is positioned in the rack with the other.

35. Process of application procedures through cell biology for animal use, according to claim 1 or 34, wherein there is the removing of all the supernatant of the four conical tubes, dispensing it; then, re-suspending the pellet of each of the tubes with 3 ml of stabilized erythrolysis medium.

36. Process of application procedures through cell biology for animal use, according to claim 1 or 35, wherein there is the transferring of the contents of each of the four conical tubes into the fifth tube, already positioned in the racks, being this tube already positioned in the centrifuge and centrifuged for a period of 5 minutes.

37. Process of application procedures through cell biology for animal use, according to claim 1, wherein there are the washing of stem cells and removal of debris include, at the end of this period, the removal of the conical centrifuge tube and placement in an appropriate rack in laminar flow.

38. Process of application procedures through cell biology for animal use, according to claim 1 or 37, therein there are the removal of the supernatant of the conical tube, dispensing it; then, re-suspending the pellet of the tube with 4 ml of the stabilized MSC-Ad medium.

39. Process of application procedures through cell biology for animal use, according to claim 1 or 38, wherein it is placed in the conical centrifuge tube, and centrifuged for a period of 3 minutes.

40. Process of application procedures through cell biology for animal use, according to claim 1 or 39, wherein, at the same shelf, it is placed a sterile 15 ml conical tube, opening the lid on it and placing the filter system with 80 ul and 10 ul pores.

41. Process of application procedures through cell biology for animal use, according to claim 1 or 40, wherein it is transferred to the tube containing the adipose tissue to the filter system; at the end of the passage, washing the filter system with more 4 ml of MSC-Ad medium.

42. Process of application procedures through cell biology for animal use, according to claims 1, 37 or 38, 39, 40 and 41, wherein the procedures re repeated two more times.

43. Process of application procedures through cell biology for animal use, according to claim 1 or 42, wherein there is the filling of stem cells into 1.0 ml syringe requires, at the end of this period, the withdrawal of conical centrifuge tube and proper positioning in a rack in a laminar flow.

44. Process of application procedures through cell biology for animal use, according to claim 1 or 43, wherein there is the removal of the supernatant of the conical tube, dispensing it.

45. Process of application procedures through cell biology for animal use, according to claim 1 or 44, therein there is the positioning of two cryovials of 1.8 ml in a rack, transferring 1.5 ml of the MSC-Ad medium for each cryovial, then re-suspending the pellet with 1.0 ml of the MSC-Ad medium.

46. Process of application procedures through cell biology for animal use, according to claim 1 or 45, wherein there is the manual homogenization of the medium with the cells, removing 0.3 ml and transferring this volume for each 1.8 ml cryotube, allowing the remaining volume in the tube. Keep it on a shelf proper.

47. Process of application procedures through cell biology for animal use, according to claim 1 or 46, wherein there is homogeneous content (using MSC-Ad and Stem Cells) of cryotubes.

48. Process of application procedures through cell biology for animal use, according to claim 1 or 47, wherein they are coupled six 30×8 disposable needles in six syringes of 1.0 ml.

49. Process of application procedures through cell biology for animal use, according to claim 1 or 48, wherein there is the filling of 0.5 ml of cryotubes contents into the 1.0 ml syringes; after finished the filling of these syringes, placing it inside a plastic content, to be inserted and sealed within a thermal reservoir to be protected in voids with cushions.

50. Process of application procedures through cell biology for animal use, according to claim 1, wherein there is the Initiation of in vitro culture of stem cells in the vertical position by two bottles of cell culture of 25 cm² in laminar flow; identifying on the external bottom and side wall of each of the cylinders, the animal's name (or veterinary), the manipulation date, the identification ROCO and the animal species.

51. Process of application procedures through cell biology for animal use, according to claims 1 and 50, wherein it is transferred 7.5 ml of MSC-Ad medium for each cell culture bottles, mix the contents of the 15 ml conical tube in the rack with the reserved name “Erythrolysis” and the date of manufacture; transferring in equal shares the contents thereof into two bottles of cell culture; and closing the lid of each bottle.

52. Process of application procedures through cell biology for animal use, according to claim 1 or 51, wherein the cell characteristics in the microscope inverted are analyzed with Hoffmann contrast.

53. Process of application procedures through cell biology for animal use, according to claim 1 or 52, wherein the cell culture bottles are transferred into the cell culture incubator, keeping them with the lids half open.

54. Process of application procedures through cell biology for animal use, according to claim 1, wherein the feeding of the In Vitro Culture includes the feeding of the cell cultures at every two days from the beginning of cell culture, and the stem cell cultures can reach the stage of maximum cell confluence of 90%.

55. Process of application procedures through cell biology for animal use, according to claims 1 and 54, wherein all actions related to the feeding stage in vitro were performed in Laminar Flow, with the bench previously cleaned with 70% alcohol.

56. Process of application procedures through cell biology for animal use, according to claim 1 or 55, wherein the cell culture bottle CO2 incubator is removed with the lid closed before, placing it lying in a laminar flow bench.

57. Process of application procedures through cell biology for animal use, according to claim 1 or 56, wherein the MSC-Ad tube is placed on a rack.

58. Process of application procedures through cell biology for animal use, according to claim 1 or 57, wherein all MSC-Ad medium contained in the cell culture bottle is removed, discarding the contents.

59. Process of application procedures through cell biology for animal use, according to claim 1 or 58, wherein it is added 8.0 ml of MSC-Ad medium into a cell culture bottle;
closing the lid and positioning the cell culture bottle so that the cellular layer is submerged in the MSC-Ad medium; returning the cell culture bottle to an incubator keeping its lid half-open.

60. Process of application procedures through cell biology for animal use, according to claim 1, wherein the Preparation of Material for freezing requires the Stabilization of Cells Freezer, composed of a tank with insulation and a shelf

61. Process of application procedures through cell biology for animal use, according to claims 1 and 60, wherein there are the transfering of liquid nitrogen from the nitrogen cylinder into the cells freezer until the specified time (about 60% of internal volume).

62. Process of application procedures through cell biology for animal use, according to claim 1 or 61, wherein the freezer with the lid is kept; after 20 minutes, check the level of the liquid nitrogen; if below the identified marking, complete with nitrogen up to the marking.

63. “Process of application procedures through cell biology for animal use” according to claim 1, wherein the labeling of the vials includes data entry of the animal and the procedure in the following order: Identification of the animal (name or RGD); species; time of freezing, print the number of labels corresponding to the number of vials to be frozen; on the bench of laminar flow, withdrawing from the package the required number of vials and tag each vial, for each vial, removing its protective paper, and next to the end with the vial bushing, fix, first, its upper edge, and then fix the rest of the label.

64. Process of application procedures through cell biology for animal use, according to claim 1, wherein the stem cell subculture (trypsinization) of stem cells in vitro culture occurs until one gets a cell confluence in the culture bottle of 90%, all actions related to the rebound step in vitro culture should be performed in Laminar Flow, with the bench previously cleaned with 70% alcohol.

65. Process of application procedures through cell biology for animal use, according to claims 1 and 64, wherein there is the stabilization of the medium to be used (Trypsin/EDTA solution, MSC-Ad medium, CRYO-MSC-Ad and Fetal Bovine Serum), at least one hour before the start of the activities of rebound and freezing, packing them in the stabilized incubator.

66. Process of application procedures through cell biology for animal use, according to claim 1 or 65, wherein the cell culture bottle in the CO₂ incubator is withdrawn with the lid previously closed, placing it lying on the laminar flow bench.

67. Process of application procedures through cell biology for animal use, according to claim 1 or 66, wherein there is the positioning of a rack in the tubes as the appropriate trypsin/EDTA solution and Fetal Bovine Serum, removed all MSC-Ad medium contained in cell culture bottle and discard the contents.

68. Process of application procedures through cell biology for animal use, according to claim 1 or 67, wherein it is added 5.0 ml of Trpsina/EDTA to the cell culture bottle, with lid closed and the bottle placed in cell culture so that the cell layer is submerged in the Trpsina/EDTA solution.

69. Process of application procedures through cell biology for animal use, according to claim 1 or 68, wherein there is the returning of the cell culture bottle to an incubator, keeping it for a period of two minutes.

70. Process of application procedures through cell biology for animal use, according to claim 1 or 69, wherein there is the evaluation, under ambient light, of the level of cell detachment from cell culture plate, being such assessment confirmed in the inverted microscope with Hoffmann contrast.

71. Process of application procedures through cell biology for animal use, according to claim 1 or 68, 69, 70, wherein the case observed that almost all stem cells came off from the cell culture plate, returning it to the laminar flow bench; if there is still a high number of cells adhered to the back plate to the incubator, keeping it for extra 30 seconds; repeating the procedures of claims 68-70.

72. Process of application procedures through cell biology for animal use, according to claim 1 or 71, wherein the cell culture bottle is positioned vertically, opening the lid and adding 5.0 ml of serum Fetal Bovine; mixing the contents of the cell culture bottle and transferring the contents of the cell culture bottle into a 15 ml conical tube.

73. Process of application procedures through cell biology for animal use, according to claim 1 or 72, wherein the tube is positioned in a 15 ml conical centrifuge and starting centrifugation, for a period of two minutes; at the end of this period, removing the conical tube from centrifuge and placing it on a rack in appropriate laminar flow, remove all the supernatant from the conical tube and dispose it.

74. Process of application procedures through cell biology for animal use, according to claim 1 or 73, wherein the pellet of the conical tube is re-suspended with 5 ml of stabilized CRYO-MSC-Ad medium, followed by homogenization.

75. Process of application procedures through cell biology for animal use, according to claim 1 or 74, wherein it is fitted in the end of the labeled vial containing the bushing to the filling syringe adapter; with the other end immersed in the CRYO-MSC-Ad medium+stem cells, and the aid of the filling syringe, aspirate the contents filling the vial, noting the fact that the last column using CRYO-MSC-Ad medium+stem cells should soak the bushing; detach the vial from the filling syringe and seal the free end of the vial.

76. Process of application procedures through cell biology for animal use, according to claim 1 or 75, wherein there is the repeating of the procedures of filling for each of the labeled vials, moving all vials filled for the freezer cell shelf; open the lid of the freezer and quickly position the shelf with the vials on the liquid nitrogen of the freezer; one must close the freezer again, keeping it closed for 20 minutes.

77. Process of application procedures through cell biology for animal use, according to claim 1 or 76, wherein, at the end of this period, if one opens the freezer and turn quickly the shelf so the vials fall directly into the liquid nitrogen; immersed in liquid nitrogen and with the aid of a tweezers; pack the vials containing frozen stem cells in the due identified racks.

78. Process of application procedures through cell biology for animal use, according to claim 1 or 77, wherein racks identified for transfer of the nitrogen cylinder, recording the information regarding the location of the racks in the cylinder (cylinder identification and the mug numbers), being the location information recorded.

79. Process of application procedures through cell biology for animal use, according to claim 1, wherein there is the heating of the water bath made with the aid of a water heater and a container (500 ml), heating 500 ml of filtered water at a temperature of 37° C.

80. Process of application procedures through cell biology for animal use, according to claim 1, wherein the preparation of cell culture bottles requires two vertical positioning in cell culture bottles in the laminar flow of 25 cm²; with a permanent marker pen, identifying the external wall and bottom side of each of the cylinders, placing the animal's name (or veterinary), the manipulation date, R1C1 identification and the animal species.

81. Process of application procedures through cell biology for animal use, according to claims 1 and 80, wherein it is transferred 7.5 ml of MSC-Ad medium for each cell culture bottle.

82. Process of application procedures through cell biology for animal use, according to claim 1, wherein the thawing of stem cells where all the action steps related to the subsequent heating of water for thawing the stem cells are held in Laminar Flow, with the bench previously cleaned with 70% alcohol, and one should find the rack that contains the cells to be thawed and check the data described in the racks and the vial.

83. Process of application procedures through cell biology for animal use, according to claims 1 and 82, wherein a vial of racks is removed to be thawed.

84. Process of application procedures through cell biology for animal use, according to claim 1 or 83, wherein the vial in the air is kept for five seconds, and after that time, dipping the vial in water heated to 37° C., keeping it immersed for ten seconds.

85. Process of application procedures through cell biology for animal use, according to claim 1 or 84, wherein there is the removing of the thawed vial from the container drying it out completely and cutting off the tip of the vial that contains the seal.

86. Process of application procedures through cell biology for animal use, according to claim 1 or 85, wherein the end to the filling syringe is coupled and cut the other end of the vial (bushing).

87. Process of application procedures through cell biology for animal use, according to claim 1 or 86, wherein the contents of the vial is transferred to a 15 ml conical tube containing 5.0 ml of MSC-Ad medium, being mentioned the 15 ml conical tube placed in the centrifuge, where it is centrifuged for a period of two minutes; at the end of this period, remove the conical tube from the centrifuge and place it in an appropriate rack in laminar flow.

88. Process of application procedures through cell biology for animal use, according to claim 1 or 87, wherein all the supernatant from the conical tube are removed and disposing it.

89. Process of application procedures through cell biology for animal use, according to claim 1 or 88, wherein there is the homogenizing of the contents of the conical tube in equal parts and transferring the contents of two bottles for cell culture previously prepared; close tight the lid of each bottle.

90. Process of application procedures through cell biology for animal use, according to claim 1 or 89, wherein there are the analysis of cell characteristics in inverted microscope with Hoffmann contrast.

91. Process of application procedures through cell biology for animal use, according to claims 1 and 90, wherein cell culture bottles are transferred to cell culture incubator, keeping them with the lids half-open.

92. Process of application procedures through cell biology for animal use, according to claims 1 and 91, wherein there is the induction of cell differentiation—osteogenic lineage, differentiation of stem cells (DIF medium) in osteoblast takes place when the cell confluence is identified between 75 and 90% of the culture bottle in vitro.

93. Process of application procedures through cell biology for animal use, according to claim 1 or 92, wherein it is made 10.0 ml of DIF medium for each bottle in vitro; the preparation of DIF medium is carried out in a laminar flow, to the table bench previously cleaned with alcohol 70%; preparation of the MSC-Ad medium in accordance with the instructions of the Operating Procedure P-003-ULAB, this medium will be the basis for making the cell differentiation medium (DIF medium); thawing an aliquot of DIF1, DIF2, DIF3; with the aid of the pipette pump and a 10 ml disposable serological pipette, add 10.0 ml of basic AD-MSC medium in 15 ml conical tube labeled “DIF”, using the automatic pipettor P20 and P200, add 10.0 μl of DIF1, 50.0 μl of DIF2 and 10 μl of the DIF3 into the conical tube “DIF” , within one hour, maintaining the “DIF” tube with the cap half-threaded in an CO₂ stabilization incubator; remove the cell culture bottle from the CO₂ incubator, with the lid previously closed, positioning it lying on the bench in the laminar flow; place, in a suitable rack, the tube with the DIF medium; with the aid of a pipette pump and 10 ml disposable serological pipette, carefully remove all MSC-Ad medium contained in the cell culture bottle, with the aid of the pipette pump and a new 10 ml disposable serological pipette, add 8.0 ml of DIF medium into the cell culture bottle, close the lid and place the cell culture bottle so that the cell layer is submerged in the MSC-Ad medium; return the cell culture bottle to an incubator, keep it with the lid half-open; repeat this procedure every 48 hours, for a period of 10 days.

94. Process of application procedures through cell biology for animal use, according to claim 1 or 92, wherein it is specific staining for osteogenic lineage cells.

95. Process of application procedures through cell biology for animal use, according to claim 1 or 94, wherein there is the removing of the cell culture bottle from the CO₂ incubator with the lid previously closed, the confluence and the aspect of the cell culture in the inverted microscope is checked; place the cell culture bottle on the bench near the sink, with an appropriate rack, place the 15 ml conical tubes with 8.0 ml with the PBS-1.DM solution (2 tubes), 2. Formalin 10% solution, 3. Deionized water, 4. Osteoblast staining solution, with the aid of the pump pipette and the 10 ml disposable serological pipette, the medium inside the cell culture bottle is removed, with the aid of a pipette pump and a new 10 ml disposable serological pipette, add 8.0 of DM-PBS into the cell culture bottle, and close the cover position of the cell culture bottle such that the cell layer is submerged in the DM-PBS; with the aid of a pipette pump and a 10 ml disposable serological pipette, 8.0 ml of 10% formalin added into the cell culture bottle, and close the cover position of the cell culture bottle such that the cell layer is submerged in the 10% formalin solution for a period of 30 minutes, with the aid of pipette pump and a 10 ml disposable serological pipette, to remove the 10% formalin solution contained in the cell culture bottle, with the aid of a pipette pump and a 10 ml disposable serological pipette, add 8.0 ml of staining in the cell culture bottle, and close the cover position of the cell culture bottle such that the cell layer is submerged in the stain solution for a period of 10 minutes, with the aid of the pipette pump and 10 ml disposable serological pipette, remove the staining solution contained in the cell culture bottle, with the aid of a pipette pump and 10 ml disposable serological pipette, add 8.0 ml of deionized water in the cell culture bottle, with the aid of the pipette pump and a 10 ml disposable serological pipette and remove the deionized water contained in the cell culture bottle, with the aid of the pipette pump and 10 mL disposable serological pipette, add 8.0 ml of PBS-DM in the cell culture bottle and close the cover and place the cell culture bottle such that the cell layer is submerged in the DM-PBS; it is observed in an inverted microscope with phase contrast the stained cells in red.
